(19) United States
(12) Patent Application Publication
(10) Pub. No.: US 2006/0210553 A1
(43) Pub. Date: Sep. 21, 2006

(54) Topical use of tyrosine kinase inhibitors of microbial origin to prevent and treat skin disorders characterised by excesssive cell proliferation

(75) Inventor: Carlo Pincelli, Sassuolo (IT)

Correspondence Address: YOUNG & THOMPSON, 745 SOUTH 23RD STREET, 2ND FLOOR, ARLINGTON, VA, 22202, US

(73) Assignee: Creabilis Therapeutics S.R.L., Colleretto Giacosa (IT)

(21) Appl. No.: 10/565,170

(22) PCT Filed: Jul. 23, 2003
(86) PCT No.: PCT/EP03/08077

§ 371 Date: Jan. 19, 2006

(51) Int. Cl. A61H 33/00; A61K 35/00; A61H 21/00

(52) U.S. Cl. 424/115; 607/86

(57) ABSTRACT

The present invention relates to the use of the alkaloid K252 and its analogues or derivatives to prepare topical drugs for the treatment of disorders characterised by hyperproliferation of keratinocytes.

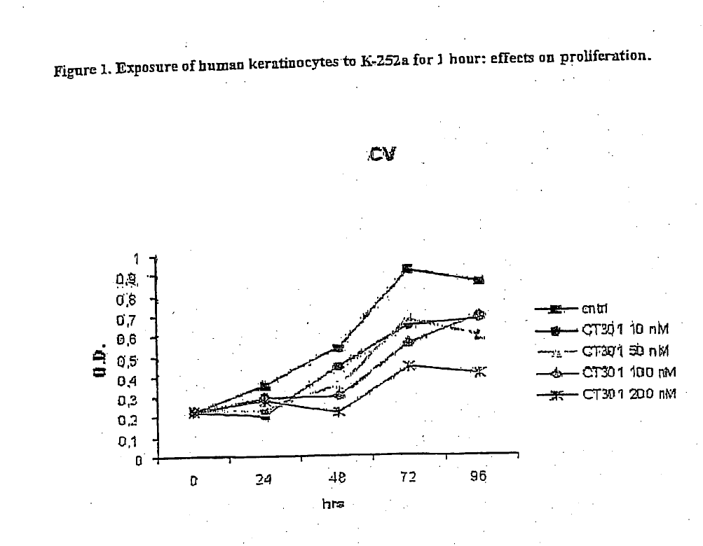

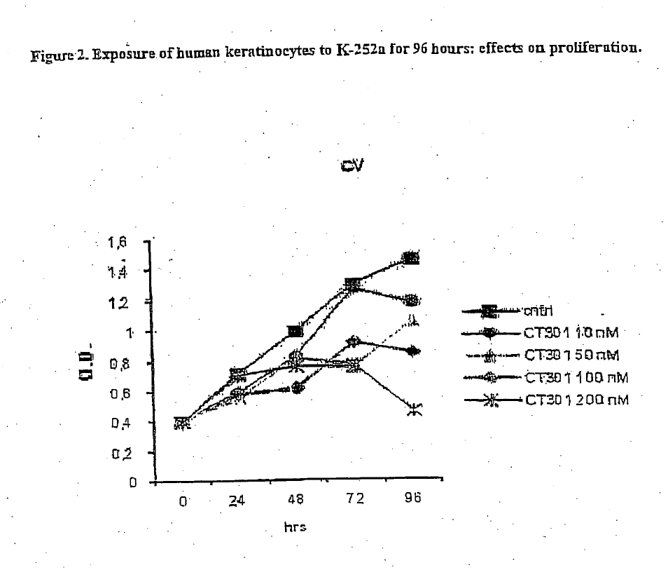

The present invention relates to the use of tyrosine kinase inhibitors of microbial origin belonging to the K252 family to prepare topical medicaments able to inhibit the excessive keratinocyte proliferation characteristic of disorders such as psoriasis and skin tumours.

### BACKGROUND OF THE INVENTION

Nerve Growth Factor (NGF) is the archetype of a family of proteins called neurotrophins (1). All members of the neurotrophin family and their receptors play a vital role in the development of the nervous system (2). In addition to this “classic” function, it is now known that NGF and the other neurotrophins are crucial molecules in modulating the inflammatory response and in tissue repair processes.

NGF acts by binding to two classes of receptors, a receptor with low affinity of ˜75 kd (p75) (3) and a tyrosine kinase receptor with high affinity of ˜140 kd (TrkA) (4). The keratinocytes express both of these receptors. NGF is released by the keratinocytes and acts in a autocrine manner on those cells.

Through binding to TrkA, autocrine NGF stimulates the proliferation of normal human keratinocyte cultures. In particular, NGF is secreted by the keratinocytes in the basal layer of the epidermis, i.e. the ones which most express TrkA. In addition to acting as mitogen, NGF also protects the keratinocytes against apoptosis (genetically programmed cell death).

The activity of the tyrosine kinase proteins seems to play a crucial role in the action mechanism of the main types of phototherapy (use of light radiation for therapeutic purposes), photochemotherapy and photodynamic treatment.

One of the main treatments for skin disorders like psoriasis and vitiligo involves the combined use of psoralens and ultraviolet light, a procedure known as PUVA treatment. This treatment profoundly alters cell growth and differentiation. In many cell types, an event that follows shortly after PUVA treatment is inhibition of the binding between EGF and its receptor through inhibition of the tyrosine kinase activity of the receptor (5). Photodynamic treatment is a recent procedure for the treatment of numerous malignant conditions, including skin tumours, involving the application of a photosensitising substance followed by illumination of the lesion with visible light. A recent study, carried out in vivo and in vitro, has demonstrated that photodynamic treatment with phthalocyanine (Pc4-PDT), which induces apoptosis in human epidermoid carcinoma cells (A431), acts by modulating the expression and phosphorylation of EGFR (6). Another study has demonstrated the efficacy of a combination of photodynamic treatment and tyrosine kinase inhibitors in inducing anti-angiogenic and anti-tumoral activity in vivo and in vitro (7).

It was recently found that an alkaloid of microbial origin, known as K252 and originally studied as an anti-allergic and antihistamine drug (U.S. Pat. No. 4,555,402), and some of its derivatives (U.S. Pat. No. 4,923,986 and U.S. Pat. No. 4,877,776), are powerful inhibitors of protein kinase C and NGF.

In particular, it has been found that K252, by inhibiting the TrkA phosphorylation induced by NGF, also inhibits the growth of human prostate carcinoma cell lines (8).

U.S. Pat. No. 6,300,327 also discloses the use of K252 and its analogues in the treatment of neurodegenerative disorders.

It has also been reported that the addition of K252 to keratinocyte cultures significantly increases both spontaneous and UV-induced apoptosis (9).

### DESCRIPTION OF THE INVENTION

It has now been found that K252 and similar compounds that inhibit the tyrosine kinase receptor of NGF can also inhibit keratinocyte proliferation.

The invention consequently relates to the use of the alkaloid K252 and its analogues or derivatives to prepare topical drugs for the treatment of disorders characterised by hyperproliferation of the keratinocytes, such as psoriasis, chonic eczema, acne, pitiriasis rubra pilaris, keloids, hypertrophic scars and skin tumours (keratoacanthoma, squamous cell carcinoma, basal cell carcinoma etc.).

Compounds K252 will be optionally used in combination with PUVA treatment or photodynamic treatment.

The invention also relates to topical pharmaceutical compositions containing an alkaloid K252 or an analogue or derivative thereof as active ingredient, in admixture with suitable vehicles and excipients.

“Alkaloid or compound K252” means the natural compounds disclosed in the above-mentioned patents, especially the compounds known as K252a and K252b, and their physiologically equivalent derivatives such as esters, amides, salts, N-alkylated or N-acylated derivatives or other derivatives obtained by chemical synthesis aimed to reduce the systemic absorption of the product, such as spacers associated to proteins or other physiologically inactive large molecules. Examples of said derivatives are disclosed in U.S. Pat. No. 4,877,776, U.S. Pat No. 4,923,986 and U.S. Pat. No. 6,300,327, the description of which is incorporated herein by reference, as is that of U.S. Pat. No. 4,555,402.

### DETAILED DESCRIPTION OF THE INVENTION

The pharmacological activity of K252 has been demonstrated by topically administering the compound directly to the skin of mice.

K252 concentrations of 50 to 500 nM in glycerin or vaseline were used.

Immunofluorescence studies demonstrated that the substance penetrates into the epidermis and the superficial dermis.

K252 was thus applied to squamous cell papillomas, induced on the skin of SENCAR and SKH-1 nude mice irradiated with UVB, once a week for 10 weeks. The treated mice presented a reduction in tumour mass of approx. 50%.

K252 was also applied on the same experimental model one hour before photodynamic treatment. The mice pre-treated with K252 required fewer sessions of photodynamic treatment than the controls.

Finally, the activity of K252, both alone and in combination with PUVA treatment, was confirmed in an experimental psoriasis model.

For the recommended therapeutic uses, K252 compounds will be formulated in pharmaceutical compositions suitable for topical administration, such as ointments, gels, lotions, powders, medicated plasters and the like, using well known techniques and excipients.

The human therapeutic dose will depend on a number of factors, and can easily be determined on the basis of pharmacotoxicological and clinical trials. Broadly speaking, concentrations of K252, its analogues or derivatives ranging from approx. 0.01% to 5% by weight of the total formulation can be used for application to the skin one or more times a day.

### REFERENCES

1. W. D. Snider, Functions of the neurotrophins during nervous system development: what the knockouts are teaching us. Cell 77 (1994), pp. 627-638.
2. G. R. Lewin and Y. A. Barde, The physiology of neurotrophins. Annu. Rev. Neurosci. 19 (1996), pp. 289-317.
3. D. Johnson, A. Lanahan, C. Randy Buck, A. Sehgal, C. Morgan and E. Mercer, Expression and structure of the human NGF receptor. Cell 47 (1986), pp. 545-554.
4. D. R. Kaplan, B. L. Hempstead, D. Martin-Zanca, M. V. Chao and L. F. Parada, The trk protooncogene product: a signal transducing receptor for Nerve Growth Factor. Science (Wash) 252 (1991), pp. 554-558.
5. Mermelstein F H. et al. Inhibition of epidermal growth factor receptor tyrosine kinase activity in A431 human epidermoid cells following psoralen/ultraviolet light treatment. Mol Pharmacol 1989; 36:848-55.
6. Ahma N. In vitro and in vivo inhibition of epidermal growth factor receptor-tyrosine kinase pathway by photodynamic therapy. Oncogene 2001; 20:2314-7.
7. Dimitroff C J. Anti-angiogenic activity of selected receptor tyrosine kinase inhibitors, PD166285 and PD173074: implications for combination treatment with photodynamic therapy. Invest New Drugs 1999; 17:121-35.
8. R. Delsite and D. Djakiew, Anti-proliferative effect of the kinase inhibitor K252a on human prostatic carcinoma cell lines. J. Androl. 17 (1996), pp. 481-490.
9. Pincelli, C, Haake, A R, Benassi, L et al. Autocrine Nerve Growth Factor protects human keratinocytes from apoptosis through its high affinity receptor (trk): a role for bcl-2. J Invest Dermatol, 109, 757-764, 1997.

1-5. (canceled)

6. Alkaloid K252 or of their physiologically equivalent derivatives selected from esters, amides, salts, N-alkylated or N-acylated derivatives or derivatives obtained by chemical synthesis aimed to reduce the systemic absorption of the product by means of spacers associated to proteins or other physiologically inactive large molecules.

7. The alkaloid K252 as claimed in claim 6, wherein the active ingredient is K252a or K252b.

8. A topical pharmaceutical composition comprising an alkaloid K252 according to claim 6, in admixture with vehicles and/or excipients.

9. The composition as claimed in claim 8 in the form of ointments, gels, lotions, powders and medicated plasters.

10. The composition as claimed in claim 8 wherein the active ingredient is K252a or K252b.

11. A method for treating hyperproliferation of keratinocytes, comprising administering an effective amount of pharmaceutical composition according to claim 8 to a subject in need thereof.

12. The method according to claim 11, further comprising administering to said subject of PUVA treatment or photodynamic treatment.

13. The method according to claim 11, wherein said subject has psoriasis and/or a skin tumour.

14. A method for preparing a pharmaceutical composition comprising adding an alkaloid K252 compound according to claim 6 with a pharmaceutical vehicle and/or excipient.
